(19) United States
(12) Patent Application Publication
(10) Pub. No.: US 2023/0087813 A1
(43) Pub. Date: Mar. 23, 2023

(54) TOOL FOR TEST BASED PERSONALIZATION OF ACTUARIAL TABLES

(71) Applicant: Assured Inc., Wellesley, MA (US)

(72) Inventors: Afik GAL, Needham, MA (US); Yariv Dror Mizrahi, Ra'anana (IL); Tal Edvabsky, Beersheva (IL); Hila Zadka-Schuldiner, Mevasseret Tzion (IL); Yehonatan Yedidia, Tel Aviv (IL)

(21) Appl. No.: 17/931,908

(22) Filed: Sep. 14, 2022

Related U.S. Application Data
(60) Provisional application No. 63/243,924, filed on Sep. 14, 2021.

(51) Int. Cl. G06Q 40/08

(52) U.S. Cl. CPC G06Q 40/08; A61B 5/4088

(57) ABSTRACT

A tool for test-based updating of actuarial tables includes an analyzer, a statistical transformer and an actuarial analyzer. The analyzer analyzes the results of cognitive and physical tests performed on a candidate of a known age and generates cognitive and physical scores for the results. The statistical transformer utilizes the scores and the known age to generate a statistical shift and generates adjusting factors Fᵢ for the actuarial tables from the statistical shift. The actuarial analyzer raises the mortality probabilities of the actuarial tables by a power of 1/Fᵢ per table, thereby to produce personalized actuarial tables for the candidate.

### CROSS REFERENCE TO RELATED APPLICATIONS

This application claims priority from U.S. provisional patent application 63/243,924, filed Sep. 14, 2021, which is incorporated herein by reference.

### FIELD OF THE INVENTION

The present invention relates to insurance generally and to underwriting of insurance policies in particular.

### BACKGROUND OF THE INVENTION

Currently, when requesting underwriting for insurance policies, customers share their historical medical records/conditions and insurance actuaries have various tables (e.g., mortality tables, disability tables, etc.) which they use to determine the costs of the insurance policies. Each row of these tables lists an age and its related probabilities, such as: the probability of a healthy person to file a claim, to die, to die if they are living in an institution, etc.

If a customer has certain conditions, like diabetes or having had cancer or being in a wheelchair, it is difficult to get insurance, since the tables indicate that such a customer is more likely to file a claim.

### SUMMARY OF THE PRESENT INVENTION

There is therefore provided, in accordance with a preferred embodiment of the present invention, a tool for test-based updating of actuarial tables. The tool includes an analyzer, a statistical transformer and an actuarial analyzer. The analyzer analyzes the results of cognitive and physical tests performed on a candidate of a known age and generates cognitive and physical scores for the results. The statistical transformer utilizes the scores and the known age to generate a statistical shift and generates adjusting factors Fᵢ for the actuarial tables from the statistical shift. The actuarial analyzer raises the mortality probabilities of the actuarial tables by a power of 1/Fᵢ per table, thereby to produce personalized actuarial tables for the candidate.

Moreover, in accordance with a preferred embodiment of the present invention, the statistical transformer includes an LE/DFLE factor determiner to generate an LE factor and a DFLE factor per a set of possible ages of candidates.

Further, in accordance with a preferred embodiment of the present invention, the LE/DFLE factor determiner includes an average time to event determiner, a shift determiner and a factor determiner. The average time to event determiner determines an average time to an insurance claim event (ATE). The shift determiner utilizes the ATE, the scores and correlations to determine the statistical shifts due to the scores and the factor determiner generates the adjusting factors Fᵢ.

Still further, in accordance with a preferred embodiment of the present invention, the insurance claim event is death or incidence of a disability.

Finally, in accordance with a preferred embodiment of the present invention, the average time to event determiner utilizes a portion of data of the actuarial tables.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a tool for personalizing actuarial tables based on test results, constructed and operative in accordance with a preferred embodiment of the present invention;

FIG. 2A is a tabular illustration of tests for early detection of cognitive decline, useful for the tool of FIG. 1;

FIG. 2B is a schematic illustration of a modified TUG test, useful for the tool of FIG. 1;

FIG. 3A is a tabular illustration of an exemplary mortality table,

FIG. 3B is a graphical illustration of the effect of an adjusting factor on a base mortality curve, useful in understanding the operation of the tool of FIG. 1;

FIG. 4A is a block diagram illustration of a LE/DFLE factor determiner, forming part the tool of FIG. 1;

FIG. 4B is a graphical illustration of an exemplary division of test result percentiles for a population aged 65 to 69, useful for the tool of FIG. 1;

FIG. 5A is a tabular illustration of a portion of an LE table listing the age, and the associated male, female and combined probabilities for that age; and

FIGS. 5B and 5C are graphical illustrations of a survival function and its inverse death function, useful in understanding the determiner of FIG. 4A, useful for the tool of FIG. 1.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that the prior art uses a candidate's past medical records to predict a future state of the candidate and from this, to make underwriting decisions. Applicant has realized that a different underwriting mechanism, based on looking forward, is possible.

Applicant has also realized that there are standardized screening tests which test a candidate's physical and cognitive abilities, and which clinical research indicates provides information about what may happen in the future. Moreover, the tests provide significantly more information about the candidate than achieved through asking questions of the candidate. Applicant has also realized that these results may affect expected mortality rate and/or likelihood of a claim and that the results may be used to adjust the current actuarial tables for each candidate, enabling candidates who currently can't get any insurance to get some kind of insurance.

However, clinical studies do not come with actuarial mortality tables but with other measures of risk, such as odds ratio and relative risk, and thus, need to be statistically translated to generate mortality curves to represent the results of these studies.

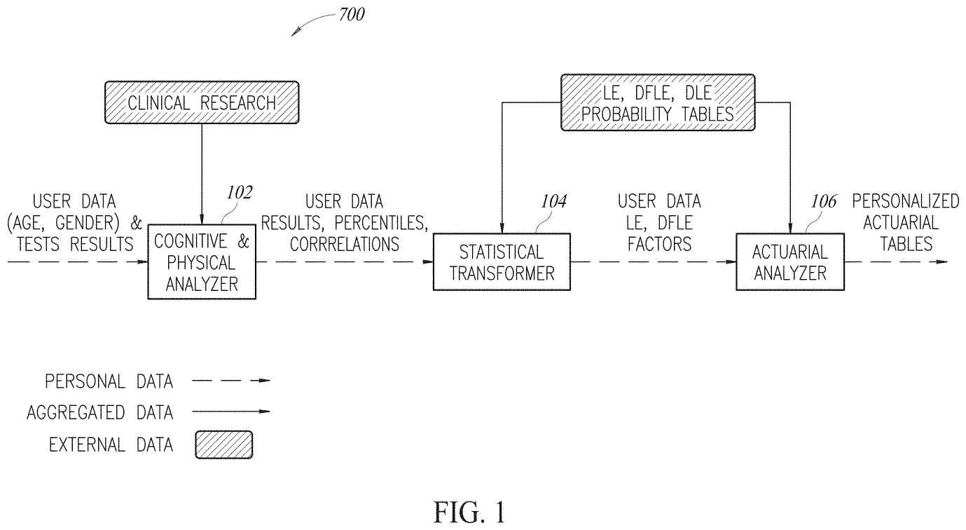

Reference is now made to FIG. 1, which illustrates a tool 100 for personalizing actuarial tables based on test results. Tool 100 may comprise an analyzer 102 and a statistical transformer 104 and may provide personalized actuarial tables 105 to an actuarial analyzer 106.

Analyzer 102 may receive data (such as gender and age) about a current candidate as well as the scores for the current candidate from tests testing his/her cognitive and physical ability. For example, the cognitive tests may be tests for early detection of cognitive decline, as described in more detail hereinbelow. The physical tests may be tests that are predictive of frailty, disability and/or mortality, as described in more detail hereinbelow

Analyzer 102 may compare the cognitive and physical scores against a table of scores, where each score value may indicate what percentage of the population also achieves that score (i.e., a percentile), thereby to generate cognitive and physical score percentiles. Analyzer 102 may find an associated correlation factor which relates test scores to claim likelihood at a given age and may generate therefrom a set of cognitive correlations and a set of physical correlations, as described in more detail hereinbelow. Typically, analyzer 102 may utilize a table relating test scores to claim likelihood to find the associated correlation.

Statistical transformer 104 may convert the output of analyzer 102 into shifts to be made to the standard actuarial tables for this candidate's age and gender. The standard actuarial tables are DFLE (disease free life expectancy), LE (life expectancy), and DLE (disease life expectancy given an event of an insurance claim) and they list, for each age of a candidate and for each gender, the expected age when a claim occurs. The shifts are the number of years by which the expected age should change, for the same gender and age, given the results on the cognitive and physical tests.

Actuarial analyzer 106 may combine the actuarial tables with the shifts for the current candidate to generate a personalized actuarial table for the current candidate, for use by an insurance company to select an appropriate plan for the current candidate based on the shifted actuarial tables. The result may be a personalized insurance plan for the current candidate.

It will be appreciated that tool 100 may connect the physical and cognitive tests, the LE, DLE and DFLE tables, and the SOA (Society of Actuaries) tables which list information relating DLE and reasons for a claim. In particular, tool 100 may adjust the LE, DFLE and DLE tables to accommodate a shift which may be a function of the correlation value related to the candidate's test scores. The correlations may be generated based on the distributions of test scores of a plurality of people who take the tests.

For example, for a 75-year-old male whose physical score may be in the lowest decile, the correlation factor may be 0.13. The shift for the LE will be calculated by the following equation:

LEshift=0.13*(LE(75,male)−LE(75,male,10th percentile))

Similar shifts may trivially be extrapolated for the candidate for the DFLE and DLE tables as well. A similar type of shifting may be implemented for other types of scores and their correlations. It is important to note that negative correlations are optional. The shifts may be utilized to define a personalized factor F with which to adjust the actuarial tables.

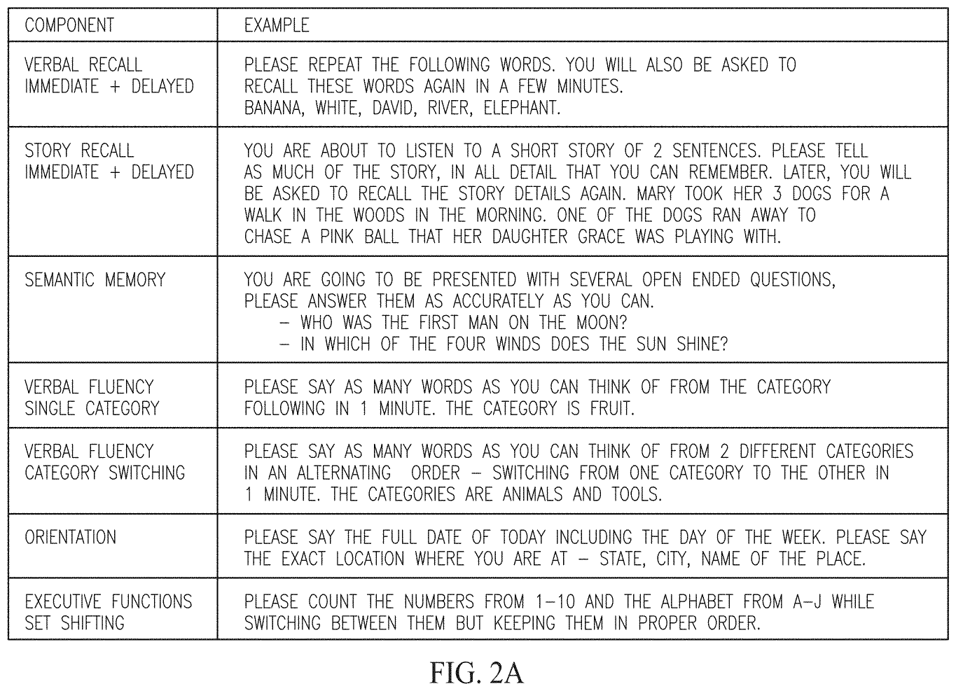

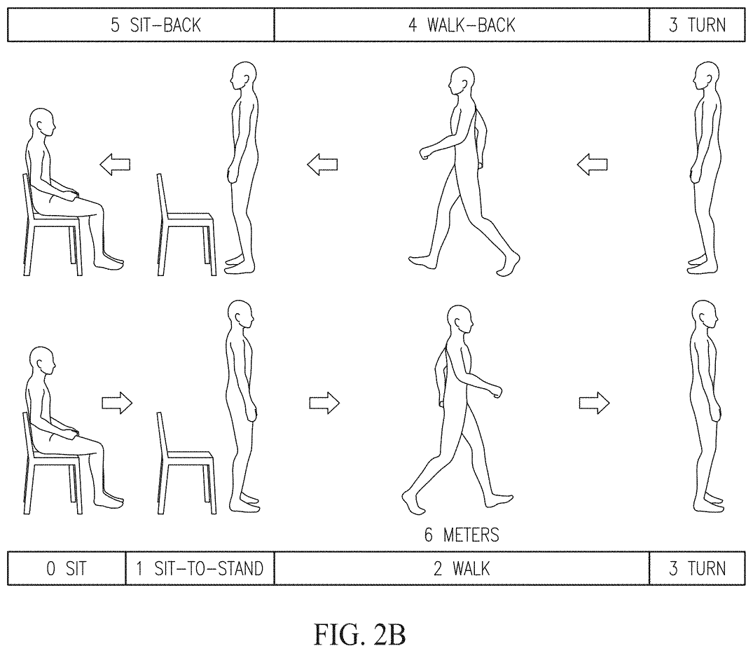

The following tests, shown in more detail in FIG. 2A to which reference is now made, may be utilized for early detection of cognitive decline:
Short term verbal memory;
Short term story recall;
Long term semantic memory;
Verbal fluency;
Cognitive shifting; and
Orientation.

The tests above are discussed and validated in the following articles to predict mild cognitive impairment (MCI), Alzheimer's disease and the progression between them:
Breton A, Casey D, Arnaoutoglou N A. Cognitive tests for the detection of mild cognitive impairment (MCI), the prodromal stage of dementia: Meta-analysis of diagnostic accuracy studies. International journal of geriatric psychiatry. 2019 February; 34(2):233-42.
Mansbach W E, Mace R A. A comparison of the diagnostic accuracy of the AD8 and BCAT-SF in identifying dementia and mild cognitive impairment in long-term care residents. Aging, Neuropsychology, and Cognition. 2016 Sep. 2; 23 (5):609-24.
Ozer S, Noonan K, Burke M, Young J, Barber S, Forster A, Jones R. The validity of the Memory Alteration Test and the Test Your Memory test for community-based identification of amnestic mild cognitive impairment. Alzheimer's & Dementia. 2016 Sep. 1; 12(9):987-95.

Tool 100 may also receive information from additional measures of cognitive performance, such as cued recall, recall of “new” items, and response times to the questions asked.

The Timed-Up-and-Go (TUG) test has been shown to be to be predictive of the risk of frailty, disability and mortality in older adults. In the TUG test, the user has to move from a sitting position to a standing position, walk a set distance, turn around, walk back to the chair and then sit down.

The TUG test is discussed and validated in the following articles to predict disability, death and their common disability risk factors:
Li T, Chen J, Hu C, Ma Y, Wu Z, Wan W, Huang Y, Jia F, Gong C, Wan S, Li L. Automatic timed up-and-go sub-task segmentation for Parkinson's disease patients using video-based activity classification. IEEE Transactions on Neural Systems and Rehabilitation Engineering. 2018 Oct. 12; 26 (11):2189-99.
Clegg A, Rogers L, Young J. Diagnostic test accuracy of simple instruments for identifying frailty in community-dwelling older people: a systematic review. Age and ageing. 2014 Oct. 29; 44(1):148-52.
Auyeung T W, Lee J S, Leung J, Kwok T, Woo J. The selection of a screening test for frailty identification in community-dwelling older adults. The journal of nutrition, health & aging. 2014 Feb. 1; 18(2):199-203.
Montero-Odasso M, Muir S W, Hall M, Doherty T J, Kloseck M, Beauchet O, Speechley M. Gait variability is associated with frailty in community-dwelling older adults. Journals of Gerontology Series A: Biomedical Sciences and Medical Sciences. 2011 May 1; 66(5):568-76.
Schwenk M, Mohler J, Wendel C, Fain M, Taylor-Piliae R, Najafi B. Wearable sensor-based in-home assessment of gait, balance, and physical activity for discrimination of frailty status: baseline results of the Arizona frailty cohort study. Gerontology. 2015; 61(3):258-67.

Tool 100 may receive data from a modified TUG test, shown in FIG. 2B to which reference is now made, which uses a larger walking distance than the standard TUG test. The patient may be videoed during the walking portion, from which gait characteristics, such as gait speed, gait variability and stride length, may be measured using existing automatic tools which review videos of people walking.

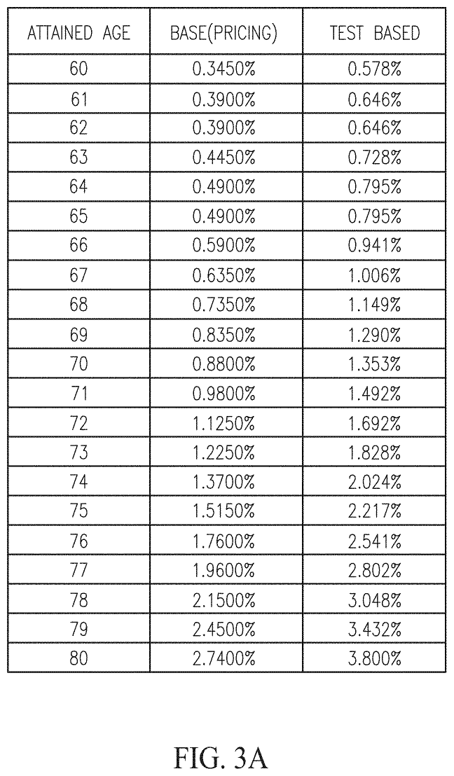

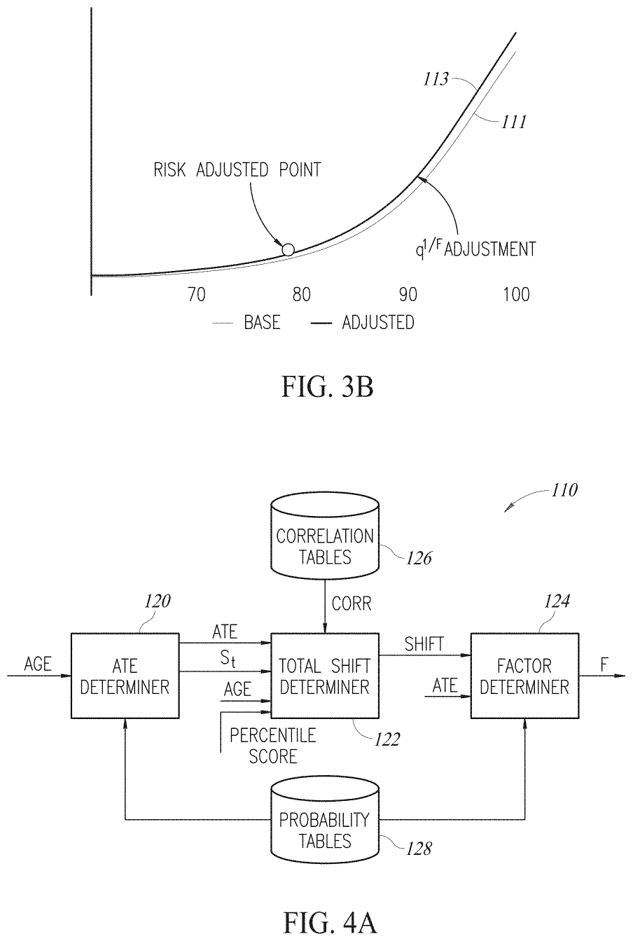

It will be appreciated that, in an ideal world, a life expectancy (LE) curve for the general population could be built from the results of a “perfect” cognitive and/or physical test (i.e. a test which predicts all the reasons for mortality and will therefore predict 100% of the future mortality in the sub-population that failed the perfect test). FIG. 3A shows an exemplary mortality table, which lists attained ages and what percentage of the population at each age is expected to die that year. FIG. 3A also shows the percentages as predicted by a test. As can be seen, the test percentages are higher and therefore, do not perfectly predict the mortalities of the population. Instead, each test only identifies some of the reasons a person might die (e.g., dementia, severe respiratory or cardiovascular failure, etc.); there might be other reasons a person might die (e.g., accidents). Thus, the mortality projections of each test are correlated only up to a certain percent, say X %. For each test, X has to be determined.

Applicant has realized that the relative risk of each participant may be measured by estimating an adjusting factor F (which is a unit-less number) for each configuration of age group, cognitive score, and physical score, and for each actuarial assumption, as discussed hereinbelow, and then to raise the mortality probabilities (i.e. the base actuarial probabilities of FIG. 3A) by a power of 1/F. It is noted that a value of F between 0 and 1 decreases the probability while a value of F greater than 1 increases the probability. The adjusting factor may be designed to impact the base actuarial assumptions, or probabilities, in such a way as to represent the participant's age, cognitive score and physical score.

The effect of the adjusting factor F is shown in FIG. 3B, to which reference is now briefly made. FIG. 3B shows a base mortality curve 111 which may be adjusted via the exponent 1/F to produce an active mortality curve 113, starting at the present year, shown as a risk adjusted point. In FIG. 3B, active mortality curve 113 is above base curve 111 indicating a lower risk of mortality. The adjusting factor may help define the effect of the cognitive and physical scores on the average mortality age. For example, males at age 60 may have, according to LE table, a 0.345% probability of dying, which may translate into an average mortality age of 87. However, if a particular male has very high physical and cognitive scores, we might expect him to live (on average) until an age of 92. In other words, due to his scores, he will probably live 5 years longer. These extra years are a “shift” in mortality. This shift is the sum of the cognitive shift (the contribution of the cognitive score) and the physical shift (the contribution of the physical score), neglecting the correlation between them.

Tool 100 may utilize the adjusting factor to change the participant's mortality probabilities in order for him to get an average age of 92 instead of 87, by raising the mortality probabilities by the power of 1/F.

For statistical transformer 104, the probabilities are divided into 3 categories, the probability to die before a claim (healthy mortality or LE (life expectancy)), the probability to claim (DFLE (disability free life expectancy)), and the probability to die during a claim (disabled mortality or DLE (disabled life expectancy)), where each category may have its own adjusting factor operative on its associated actuarial table.

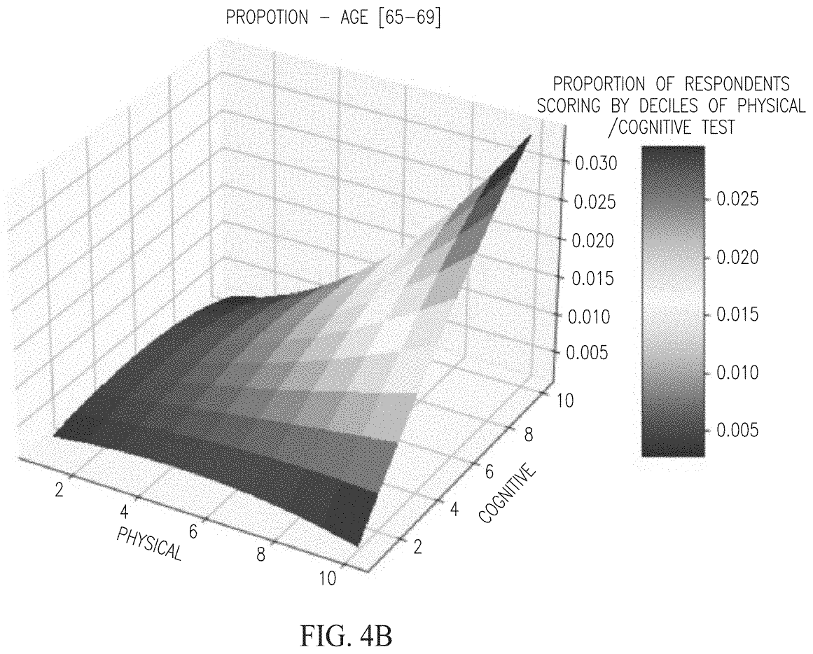

Reference is now made to FIG. 4A, which illustrates a LE/DFLE factor determiner 110, forming part of statistical transformer 104, which may generate the life expectancy and disability free life expectancy factors. LE/DFLE factor determiner 110 may comprise an ATE (average time to event) determiner 120, a total shift determiner 122 and a factor determiner 124, and which may utilize data of probability or actuarial tables 128 and correlation tables 126.

Statistical transformer 104 may compute the LE and DFLE adjusting factors apriori for each age group and a pair of a cognitive and physical score percentiles or deciles (i.e., scores within a bracket of 10 percentiles (e.g., the 7^{th} decile has scores from 70-79 in it)). For example, there may be 5 age groups and 100 pairs of score deciles, resulting in 500 adjusting factors to be computed.

FIG. 4B, to which reference is now made, shows an exemplary division of test result percentiles for a population aged 65 to 69, generated from the NHATS (National Health and Aging Trends Study) data of 2011. Since different people were in the study for different lengths of time and at different ages, each person's results were weighted by number of years that person was in the study. For this population, the physical tests were part of the short physical performance battery and the cognitive tests were verbal recall, clock drawing, semantic memory. For each age group, the joint distribution of the physical scores and the cognitive scores was calculated and divided into deciles. As can be seen, there are more people with lower scores (i.e., in the lower percentiles) than in the higher percentiles.

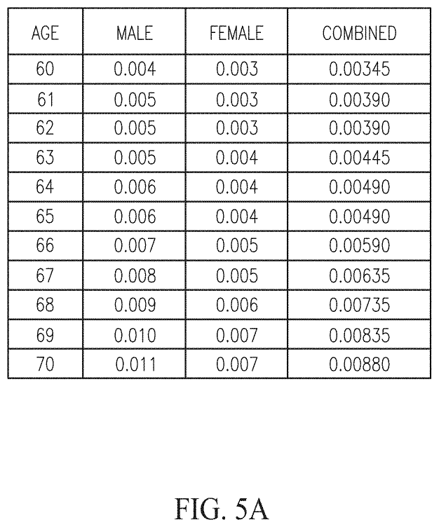

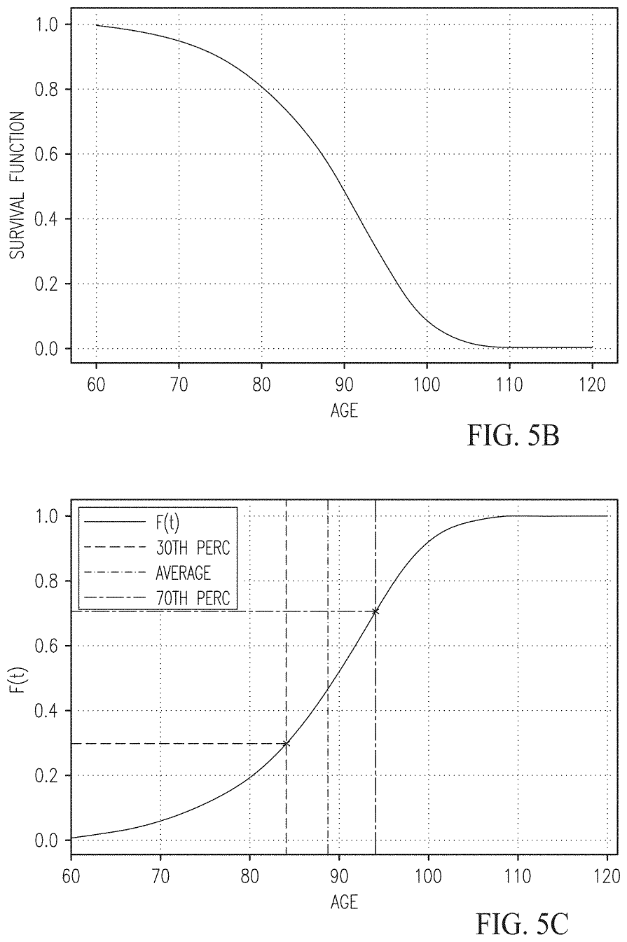

ATE determiner 120 may determine an average time to an event for which a claim may be made, such as for death (LE) or incidence of a disability (DFLE). To do so, ATE determiner 120 may receive an age of the candidate for insurance and may select a set of T next probabilities from the relevant LE or DFLE table in probability tables 128, where T may be 30. FIG. 5A, to which reference is now briefly made, shows a portion of an LE table listing the age, and the associated male, female and combined probabilities for that age. The combined column is a weighted sum between the male and female columns, using 45% weight for males. For example, the probability that a male aged 63 might die in the next year is 0.005 (i.e., 0.5%), that a woman might die is 0.0003 and that a person at aged 63 might die is 0.0039.

Let's denote these probabilities by pᵢ where i indicates the year following the current age. To compute his average time to the event, ATE determiner 120 may first estimate a survival function S(t) which defines the probability of the candidate to survive t years from now. This which is the probability that the candidate didn't die in year 1 AND didn't die in year 2, AND so on. The survival function S(t) at any time t may be defined as follows:

S(t)=(1−p₁)*(1−p₂)* . . . *(1−pₜ) (1)

S₀=1 (2)

The survival function S(t) is a decreasing function, as can be seen in FIG. 5B, to which reference is now briefly made. The exemplary survival function S(t) in FIG. 5B was calculated from the LE probabilities from age 60 and begins with the probability at age=60, which is exactly 1, because the exemplary candidate is already at age 60, and it decreases until age 90 where it is around 50%, after which it decreases less steeply.

Applicant has realized that the integral of the survival function S(t) is the average time to event (ATE); therefore, ATE determiner 120 may then determine the average time to event (ATE) from T survival values S(t) per equation 3, as follows:

ATE=∫ₜ₌₁^{T}S(t) (3)

Total shift determiner 122 may utilize the average time to event ATE, as well as the percentile scores of the candidate on the cognitive and physical tests, and may determine a cognitive shift Shift_{cog}, due to the cognitive scores and a physical shift Shift_{phy} due to the physical scores, as well as the total shift Shiftₜₒₜ, which is a summation of both the cognitive and the physical shift and is defined in equation 4 as follows:

Shiftₜₒₜ=Shift_{phy}+Shift_{cog} (4)

The total shift Shiftₜₒₜ is the shift (in years) due to both the cognitive and the physical scores from the average years to healthy mortality, as explained in the example above. Total shift determiner 122 may estimate these two shifts, cognitive and physical, in the same manner.

Total shift determiner 122 may determine each type of shift from the difference between an average age at an event and a “percentile” age for that event (i.e., the age at the event for the specific percentile) and a correlation value defining the relative contribution of the scored test to the DFLE or the LE population, per age of candidate.

Mathematically, this is written as:

Shift_{type}=Corr_{type}*(Percentile Age at event−Average Age at event) (5)

Total shift determiner 122 may determine the average age at event as the starting age plus the average time to event ATE:

Average Age at event=Age+ATE (6)

Total shift determiner 122 may determine the percentile age at event from the survival function S_{T} and the percentile score for the specific type (cognitive or physical). Initially, total shift determiner 122 may convert survival function S(t) into a death function F(t) which is the probability to die at time t, as follows:

F(t)=1−S(t) (7)

Death function F(t) is shown in FIG. 5C, to which reference is now made, as a solid line. It indicates the percentage of people that died up to time t. FIG. 5C also indicates the age at death for the average person, at age 88, and for the 30^{th} percentile and the 70^{th} percentile, as 84 and 94, respectively.

Total shift determiner 122 may find the time t on death function F(t) that matches the candidate's percentile score on the cognitive or physical tests. FIG. 5C shows that 70% of the population has died by age 94 and thus, if the candidate received a 70^{th} percentile score on a test, the percentile age to the event of the test is 6 years above the average of 88. FIG. 5C also shows that, if the candidate received a 30^{th} percentile score on a test, the percentile age at the event is 84 (i.e., −4 years to the event).

Total shift determiner 122 may determine the time until event for the specific percentile as:

Percentile time to event=argminₜ(abs(F(t)−F(t) at the percentile age)) (8)

and may determine the percentile age at event as:

Percentile Age at event=Age+Percentile time to event (9)

Total shift determiner 122 may pull the relevant correlation coefficients, for the relevant type of test and for the age of the candidate, from correlation tables 126. Correlation tables 126 may store the relative contribution of the scored test to the DFLE or the LE population, per age of candidate.

For example, the expected time for a claim might be 10 years and a participant may score in the 80% percentile on his cognitive test. From the DFLE distribution, the value for the 80^{th} percentile is 16 years, so the shift would seem to be 6 years. However, according to literature of the Society of Actuaries, only 30% of the claims are related to cognitive issues (dementia, Alzheimer's). So only 30% of the 6 years of shift are explained by the cognitive test and thus, only 1.8 years of a shift are related to the cognitive test. Correlation tables 126 may then list a correlation of 0.3.

An exemplary correlation table 126 may be:

| | LE | LE | DFLE | DFLE |
|---|---|---|---|---|
| Age | Cognitive | Physical | Cognitive | Physical |
| 60 | 0.077 | 0.21 | 0.27 | 0.12 |
| 65 | 0.075094154 | 0.21 | 0.298101817 | 0.11126598 |
| 70 | 0.07323548 | 0.21 | 0.329128493 | 0.103167653 |
| 75 | 0.071422811 | 0.21 | 0.363384451 | 0.09565875 |
| 80 | 0.069655007 | 0.21 | 0.401205797 | 0.088696372 |

Total shift determiner 122 may then compute the per type shift, Shift_{type} according to equation 4, using the results from equations 6 and 9, and then may determine the total shift Shift_{Tot} from the per type shifts.

Factor determiner 124 may compute the factors F_{LE} and F_{DFLE} for the LE and DFLE categories for raising the healthy mortality probabilities to get an adjusted, average time to event, ATE′, defined as:

ATE′=ATE+Shiftₜₒₜ (10)

Applicant has realized that the adjusted average time to event is just the integral of an adjusted survival function S(t)′, as follows:

ATE′=integral S(t)′=ATE+Shiftₜₒₜ (11)

where the adjusted survival function S(t)′, following equation 1, is defined as:

S(t)′=(1−p₁′)*(1−p₂′)* . . . *(1−pₜ′) (12)

and the adjusted percentages pᵢ′ are adjusted by the relevant adjusting factor F, as follows:

pᵢ′—pᵢ^{(1/F)} (13)

Factor determiner 124 may compute the value of the adjusting factor F given the value of ATE′ from equation 10 and solving for the factor F using equations 12 and 13. Factor determiner 124 may compute the adjusting factor F for each age, and combination of score percentiles, for both the LE and the DFLE probability tables 128.

It will be appreciated that actuarial analyzer 106 may utilize the LE/DFLE factors produced by statistical transformer 104 to produce the relevant personalized actuarial table for the candidate. Actuarial analyzer 106 may do so by raising the mortality probabilities of each actuarial table with its relevant factor F_{LE/DFLE} by a power of 1/F.

With the personalized actuarial table, an insurance company may determine which of its many different insurance plans is best suited for the candidate.

Unless specifically stated otherwise, as apparent from the preceding discussions, it is appreciated that, throughout the specification, discussions utilizing terms such as “processing,” “computing,” “calculating,” “determining,” or the like, refer to the action and/or processes of a general purpose computer of any type, such as a client/server system, mobile computing devices, smart appliances, cloud computing units or similar electronic computing devices that manipulate and/or transform data within the computing system's registers and/or memories into other data within the computing system's memories, registers or other such information storage, transmission or display devices.

Embodiments of the present invention may include apparatus for performing the operations herein. This apparatus may be specially constructed for the desired purposes, or it may comprise a computing device or system typically having at least one processor and at least one memory, selectively activated or reconfigured by a computer program stored in the computer. The resultant apparatus when instructed by software may turn the general-purpose computer into inventive elements as discussed herein. The instructions may define the inventive device in operation with the computer platform for which it is desired. Such a computer program may be stored in a computer readable storage medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing electronic instructions and capable of being coupled to a computer system bus. The computer readable storage medium may also be implemented in cloud storage.

Some general-purpose computers may comprise at least one communication element to enable communication with a data network and/or a mobile communications network.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A tool for test-based updating of actuarial tables, the tool comprising:
an analyzer to analyze results of cognitive and physical tests performed on a candidate of a known age, said analyzer to generate cognitive and physical scores for said results;
a statistical transformer to utilize said scores and said known age to generate a statistical shift and to generate adjusting factors Fᵢ for said actuarial tables from said statistical shift; and
an actuarial analyzer to raise mortality probabilities of said actuarial tables by a power of 1/Fᵢ per table, thereby to produce personalized actuarial tables for said candidate.

2. The tool of claim 1 wherein said statistical transformer comprises an LE/DFLE factor determiner to generate an LE factor and a DFLE factor per a set of possible ages of candidates.

3. The tool of claim 2 wherein said LE/DFLE factor determiner comprises:
an average time to event determiner to determine an average time to an insurance claim event (ATE);
a shift determiner to utilize said ATE, said scores and correlations to determine said statistical shifts due to said scores; and
a factor determiner to generate said adjusting factors Fᵢ.

4. The tool of claim 3 wherein said insurance claim event is for one of: death and incidence of a disability.

5. The tool of claim 3 wherein said average time to event determiner to utilize a portion of data of said actuarial tables.
